(19) United States
(12) Patent Application Publication
(10) Pub. No.: US 2018/0117197 A1
(43) Pub. Date: May 3, 2018

(54) BACTERIA REMOVING WATER GENERATION DEVICE

(71) Applicant: TOTO LTD., Fukuoka (JP)

(72) Inventors: Masahiro Kuroishi, Kitakyushu-shi, Fukuoka (JP); Takamasa Suzuki, Kitakyushu-shi, Fukuoka (JP); Yusuke Nogoshi, Kitakyushu-shi, Fukuoka (JP); Kenta Suzuki, Kitakyushu-shi, Fukuoka (JP); Muneyuki Urata, Kitakyushu-shi, Fukuoka (JP); Koki Nagano, Kitakyushu-shi, Fukuoka (JP); Yukiko Yano, Kitakyushu-shi, Fukuoka (JP); Yusuke Nakamura, Kitakyushu-shi, Fukuoka (JP)

(73) Assignee: TOTO LTD, Fukuoka (JP)

(21) Appl. No.: 15/559,511

(22) PCT Filed: Feb. 29, 2016
(86) PCT No.: PCT/JP2016/056134

§ 371 Date: Sep. 19, 2017

(30) Foreign Application Priority Data
Mar. 20, 2015 (JP) 2015-058190

(51) Int. Cl. A61L 2/18; C02F 1/461

(52) U.S. Cl. CPC A61L 2/18; C02F 1/4618; E03C 1/02; C02F 2303/04; A61L 2202/11

(57) ABSTRACT

A bacteria removing water generation device is provided. The device includes a hypochlorous acid containing water generating part generating water containing hypochlorous acid by electrolyzing tap water; a sterilizing metal ionic water generating part including electrodes, sterilizing metal ion being eluted from the electrodes into the tap water by electrolyzing tap water; a water discharge part discharging at least one of the water containing the hypochlorous acid and water containing the sterilizing metal ion; and a controller controlling operations of the hypochlorous acid containing water generating part and the sterilizing metal ionic water generating part. When a concentration of the hypochlorous acid in the water containing the hypochlorous acid is relatively low, a concentration of the sterilizing metal ion in the water containing the sterilizing metal ion is relatively high.

### TECHNICAL FIELD

An aspect of the invention generally relates to a bacteria removing water generation device.

### BACKGROUND ART

For example, in order to remove bacteria from an object which bacteria removing water is hard to stay and much dirt exists (for example, a wash place floor of a bath room or the like) with a small amount of bacteria removing water having a low concentration (concentration suppressing influence to a human body), it is favorable to discharge water containing hypochlorous acid and water containing sterilizing metal ion to the object. For example, there is a function water generation device including a sterilizing metal addition unit adding sterilizing metal ion to supply water and a hypochlorous acid addition unit adding hypochlorous acid to supply water (Patent Document 1).

Generally, water containing hypochlorous acid has an immediate effect, and can destroy a cell membrane of bacteria in a short time, on the other hand, it is easy to react dirt extremely. Therefore, a concentration of hypochlorous acid decreases because the water containing hypochlorous acid reacts with the dirt before attacking nucleus of the bacteria.

In contrast, an immediate effect of the water containing sterilizing metal ion is lower than the immediate effect of the water containing hypochlorous acid. Therefore, the water containing sterilizing metal ion cannot destroy the cell membrane of the bacteria in a short time, however it is hard to react with the dirt in comparison with the water containing hypochlorous acid and enters cytoplasm through the cell membrane to suppress cell division of the nucleus. Therefore, a concentration of sterilizing metal ion lasts over a long time.

In this way, the water containing hypochlorous acid and the water containing sterilizing metal ion complement and comply with each other. Therefore, the water containing hypochlorous acid destroys the cell membrane of the nucleus in a short time, and the water containing sterilizing metal ion enters the inside of the bacteria from the cell membrane destroyed by the water containing hypochlorous acid, and then can attack the nucleus of the bacteria under a condition of lasting the concentration over a long time.

However, the concentration of hypochlorous acid is proportional to the amount of chlorine ion contained in tap water. Therefore, in the case where the amount of chlorine ion is small, the concentration of hypochlorous acid decreases, and destruction of the cell membrane of the bacteria may be insufficient. In the case where the destruction of the cell membrane of the bacteria is insufficient, there is a problem that the water containing sterilizing metal ion cannot arrive at the nucleus of the bacteria and a bacteria removing effect is insufficient.

### PRIOR ART DOCUMENT

### Patent Document

[Patent Document 1] Japanese publication of patent application 2001-252674 (Kokai)

### SUMMARY OF INVENTION

### Problem to be Solved by Invention

The invention has been made based on the recognition of such a problem, and an object of the invention is to provide a bacteria removing water generation device capable of exerting a sufficient bacteria removing effect even if a concentration of chlorine ion contained in tap water is low.

### Means for Solving Problem

The first invention is a bacteria removing water generation device comprising: a hypochlorous acid containing water generating part generating water containing hypochlorous acid by electrolyzing tap water; a sterilizing metal ionic water generating part including electrodes, sterilizing metal ion being eluted from the electrodes into the tap water by electrolyzing tap water; a water discharge part discharging at least one of the water containing the hypochlorous acid and water containing the sterilizing metal ion; and a controller controlling operations of the hypochlorous acid containing water generating part and the sterilizing metal ionic water generating part, when a concentration of the hypochlorous acid in the water containing the hypochlorous acid is relatively low, a concentration of the sterilizing metal ion in the water containing the sterilizing metal ion being relatively high.

Hypochlorous acid is generated from chlorine ion contained in the tap water as a raw material. Therefore, when the amount of chlorine ion contained in the tap water is relatively low, the concentration of hypochlorous acid is relatively low. On the other hand, sterilizing metal ion is generated from the electrode eluted. Therefore, the concentration of sterilizing metal ion does not depend on the amount of chlorine ion contained in the tap water. However, the generation amount of sterilizing metal ion is limited.

According to this bacteria removing water generation device, even if the concentration of chlorine ion contained in the tap water is relatively low and the concentration of hypochlorous acid is relatively low, a sufficient bacteria removing effect can be exerted. On the other hand, when the concentration of hypochlorous acid is relatively high, the concentration of sterilizing metal ion is relatively low. Thereby, while ensuring the sufficient bacteria removing effect, a life of the electrodes of the sterilizing metal ionic water generating part can be improved.

The second invention is the bacteria removing water generation device in the first invention, wherein when the concentration of the hypochlorous acid is relatively low, a change rate of the concentration of the sterilizing metal ion is relatively high.

When the concentration of hypochlorous acid is relatively high, the water containing hypochlorous acid can destroy the cell membrane sufficiently. On the other hand, when there is the dirt, the effect of the water containing hypochlorous acid to attack the nucleus of the bacteria is lower than the effect of the water containing sterilizing metal ion to attack the nucleus of the bacteria.

When the concentration of hypochlorous acid is comparatively low, the water containing hypochlorous acid may not sufficiently destroy the cell membrane. In such a case, the water containing sterilizing metal ion is also required to destroy the cell membrane of the bacteria.

According to this bacteria removing water generation device, when the concentration of hypochlorous acid is relatively low, a change rate of the concentration of sterilizing metal ion is relatively high. Thereby, even if the concentration of hypochlorous acid is relatively low, the cell membrane can be destroyed sufficiently.

The third invention is the bacteria removing water generation device in the first or second invention, wherein the concentration of the sterilizing metal ion is higher than a prescribed concentration even if the concentration of the hypochlorous acid is high.

The water containing hypochlorous acid is likely to react with the dirt in comparison with the water containing sterilizing metal ion. Therefore, even if the concentration of hypochlorous acid is high, the effect of the water containing hypochlorous acid to attack the nucleus of the bacteria is lower than the effect of the water containing sterilizing metal ion to attack the nucleus of the bacteria. When the concentration of sterilizing metal ion is lower than a prescribed concentration, the effect of the water containing sterilizing metal ion to attack the nucleus of the bacteria is low.

According to this bacteria removing water generation device, since the concentration of sterilizing metal ion is higher than a prescribed concentration, even if the concentration of hypochlorous acid is high, the effect of the water containing sterilizing metal ion to attack the nucleus of the bacteria is maintained.

The fourth invention is the bacteria removing water generation device in one of the first to third inventions, wherein the water discharge part discharges the water containing a relatively large amount of hypochlorous acid when he concentration of hypochlorous acid is relatively low.

According to this bacteria removing water generation device, even if the concentration of hypochlorous acid is relatively low, the cell membrane can be destroyed sufficiently.

The fifth invention is the bacteria removing water generation device in the fourth invention, wherein an instantaneous flow rate of the water containing the hypochlorous acid when the concentration of the hypochlorous acid is relatively low is the same as an instantaneous flow rate of the water containing the hypochlorous acid when the concentration of the hypochlorous acid is relatively high.

According to this bacteria removing water generation device, since the instantaneous flow rate of the water containing hypochlorous acid is constant, even if the concentration of hypochlorous acid is relatively low, while maintaining a state of higher concentration of hypochlorous acid, the water containing a larger amount of hypochlorous acid can be discharged. Thereby, the cell membrane of the bacteria can be more certainly destroyed.

The sixth invention is the bacteria removing water generation device in the fifth invention, wherein an increasing rate of a discharge amount of the water containing the sterilizing metal ion when the concentration of the hypochlorous acid is relatively low is lower than an increasing rate of a discharge amount of the water containing the hypochlorous acid when the concentration of the hypochlorous acid is relatively low.

An immediate effect of the water containing sterilizing metal ion is lower than the immediate effect of the water containing hypochlorous acid. Therefore, if the discharge amount of the water containing hypochlorous acid is increased, a certain effect is obtained, however even if the discharge amount of the water containing sterilizing metal ion is increased, it is impossible to obtain an effect as much as the effect obtained when the discharge amount of the water containing hypochlorous add is increased.

According to this bacteria removing water generation device, while ensuring the sufficient bacteria removing effect, the life of the electrodes of the sterilizing metal ionic water generating part can be improved.

The seventh invention is the bacteria removing water generation device in one of the first to sixth inventions, further comprising: an operation part, when the concentration of the hypochlorous acid is relatively low, in the operation part a mode being selectable, in the mode the water discharge part discharging the water containing the relatively large amount of the hypochlorous acid.

According to this bacteria removing water generation device, the user can discharge the water containing the relatively large amount of hypochlorous acid when the concentration of hypochlorous acid is relatively low from the water discharge part based on the intention of the user itself. Thereby, even if the concentration of chlorine ion contained in the tap water is low, the user can destroy the cell membrane of the bacteria certainly based on the intention of the user itself.

### Effect of Invention

According to an aspect of the invention, a bacteria removing water generation device which is capable of exerting a sufficient bacteria removing effect even if a concentration of chlorine ion contained in tap water is low is provided.

### BRIEF DESCRIPTION OF DRAWINGS

FIG. 1 is a block diagram showing a bacteria removing water generation device according to an embodiment of the invention.

FIG. 2 is a schematic cross-sectional view showing a specific example of a hypochlorous acid containing water generating part of the embodiment.

FIG. 3 is a schematic cross-sectional view showing a specific example of a sterilizing metal ionic water generating part of the embodiment.

FIG. 4 is a schematic cross-sectional view showing an action of water containing hypochlorous acid and water containing sterilizing metal ion.

FIG. 5 is a schematic cross-sectional view showing an action of the water containing hypochlorous acid.

FIG. 6 is a schematic cross-sectional view showing an action of the water containing sterilizing metal ion.

FIG. 7 is a graph view illustrating one example of the relationship between a concentration of hypochlorous acid and a concentration of sterilizing metal ion.

FIG. 8 is a flow chart showing an operation of the bacteria removing water generation device according to the embodiment.

FIG. 9 is a schematic cross-sectional view showing a bath room including the bacteria removing water generation device according to the embodiment.

FIG. 10 is a schematic enlarged view shown by enlarging a portion where the bacteria removing water generation device is provided.

### EMBODIMENTS OF INVENTION

Embodiments of the invention will now be described with reference to the drawings. Note that the same numerals are applied to similar constituent elements in the drawings and detailed descriptions of such constituent elements are appropriately omitted.

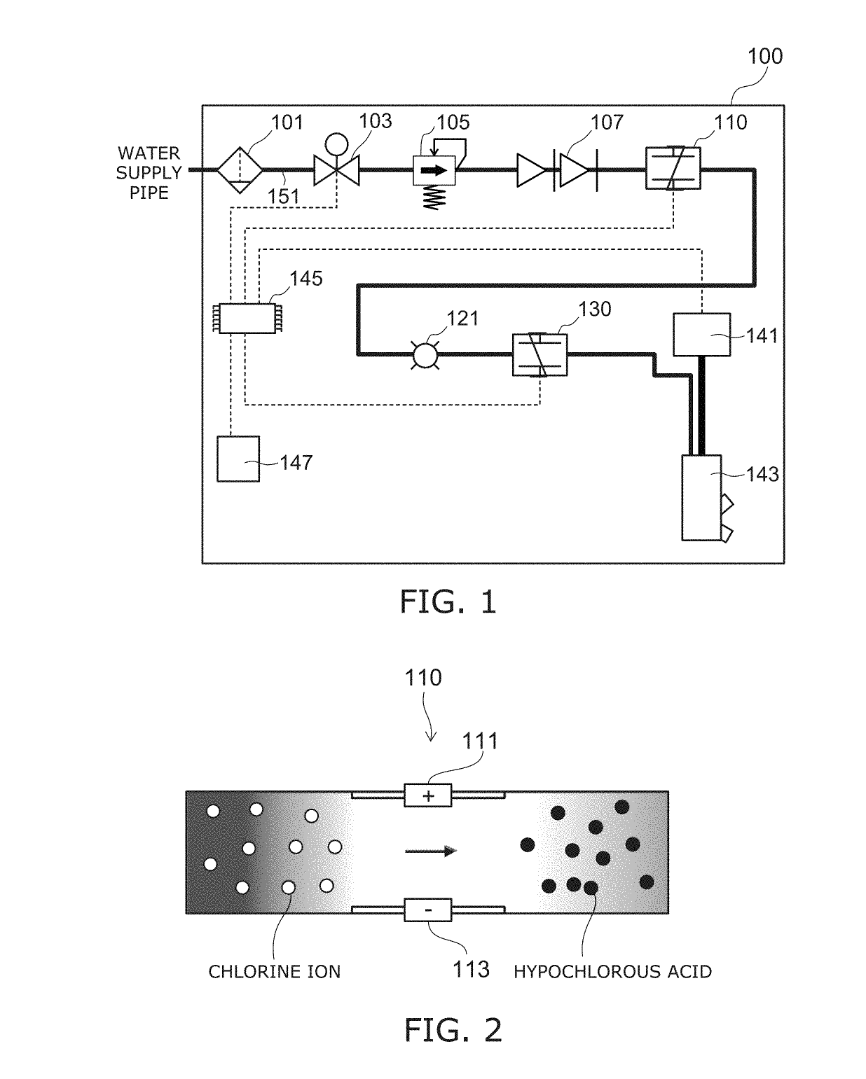

FIG. 1 is a block diagram showing a bacteria removing water generation device according to an embodiment of the invention.

FIG. 1 shows a relevant configuration of a water channel system with a relevant configuration of an electrical system.

A bacteria removing water generation device 100 according to the embodiment includes a stopcock 101, a solenoid valve 103, a pressure regulator valve 105, a check valve 107, a hypochlorous acid containing water generating part 110, a negative pressure breaking device (vacuum breaker) 121, a sterilizing metal ionic water generating part 130, a motor 141, a water discharge part 143, a controller 145, and an operation part 147.

The stopcock 101 is connected to a water supply pipe, and is openable and closable manually. For example, the stopcock 101 is possible to shut off a flow channel 151 at any time on installation/removal and maintenance and inspection or the like of the bacteria removing water generation device 100. The stopcock may be provided on the bacteria removing water generation device 100, or may be provided as a separate element between the water supply pipe and the bacteria removing water generation device 100.

The solenoid valve 103 is provided downstream of the stopcock 101. The solenoid valve 193 switches open/close state of the flow channel 151 based on a signal outputted from the controller 145, and controls water supply.

The pressure regulator valve 105 is provided on downstream of the solenoid valve 103. The pressure regulator valve 105 regulates the water supply pressure within a prescribed range when the pressure of the water (water supply pressure) supplied from the water supply pipe is high.

The check valve 107 is provided on downstream of the pressure regulator valve 105. The check valve 107 suppresses the water containing hypochlorous acid flowing backward from the hypochlorous acid containing water generating part 110 toward the water supply pipe and suppresses the water containing sterilizing metal ion flowing backward from the sterilizing metal ionic water generating part 130 toward the water supply pipe when the pressure inside the flow channel 151 decreases or so.

The hypochlorous acid containing water generating part 110 is provided downstream of the check valve 107. Here, a specific example of the hypochlorous acid containing water generating part 110 will be described with reference to the drawing.

FIG. 2 is a schematic cross-sectional view showing the specific example of a hypochlorous acid containing water generating part of the embodiment.

The hypochlorous acid containing water generating part 110 of the specific example includes an anode 111 and a cathode 113 therein, and can electrolyze the tap water flowing in a space (flow channel) between the anode 11 and the cathode 113 by the control signal of energization outputted from the controller 145. If the voltage is supplied between the anode 111 and the cathode 113, a reaction presented by the formula (1) occurs at the cathode 113.

H⁺+e⁻→½H₂↑ (1)

On the other hand, if the voltage is supplied between the anode 111 and the cathode 113, reactions represented by the formula (2) and formula (3) occur at the anode 111.

2OH⁻→2e⁻+H₂O+½O₂↑ (2)

Cl⁻→e⁻+½Cl₂ (3)

Chlorine generated in the formula (3) is hard to exist as air bubbles, and almost all chlorine dissolve in water. Therefore, with respect to chlorine generated in the formula (3), a reaction represented by the formula (4) occurs. In this way, hypochlorous acid (HCIO) is generated by electrolyzing chlorine ion. As a result, the water electrolyzed in the hypochlorous acid containing water generating part 110 changes to the water containing hypochlorous acid.

Cl₂+H₂O→HClO+H⁺+Cl⁻ (4)

Returning to FIG. 1, the negative pressure breaking device 121 is provided downstream of the hypochlorous acid containing water generating part 110. The negative pressure breaking device 121 suppresses the water containing sterilizing metal ion from blowing backward from the sterilizing metal ionic water generating part 130 toward the water supply pipe, for example, when a negative pressure is generated in the flow channel 151 or so. Or the negative pressure breaking device 121 takes in air from the outside on water drainage of the flow channel 151 and promotes the water drainage of the flow channel 151.

The sterilizing metal ionic water generating part 130 is provided downstream of the negative pressure breaking device 121. Here, a specific example of the sterilizing metal ionic water generating part 130 will be described with reference to the drawing.

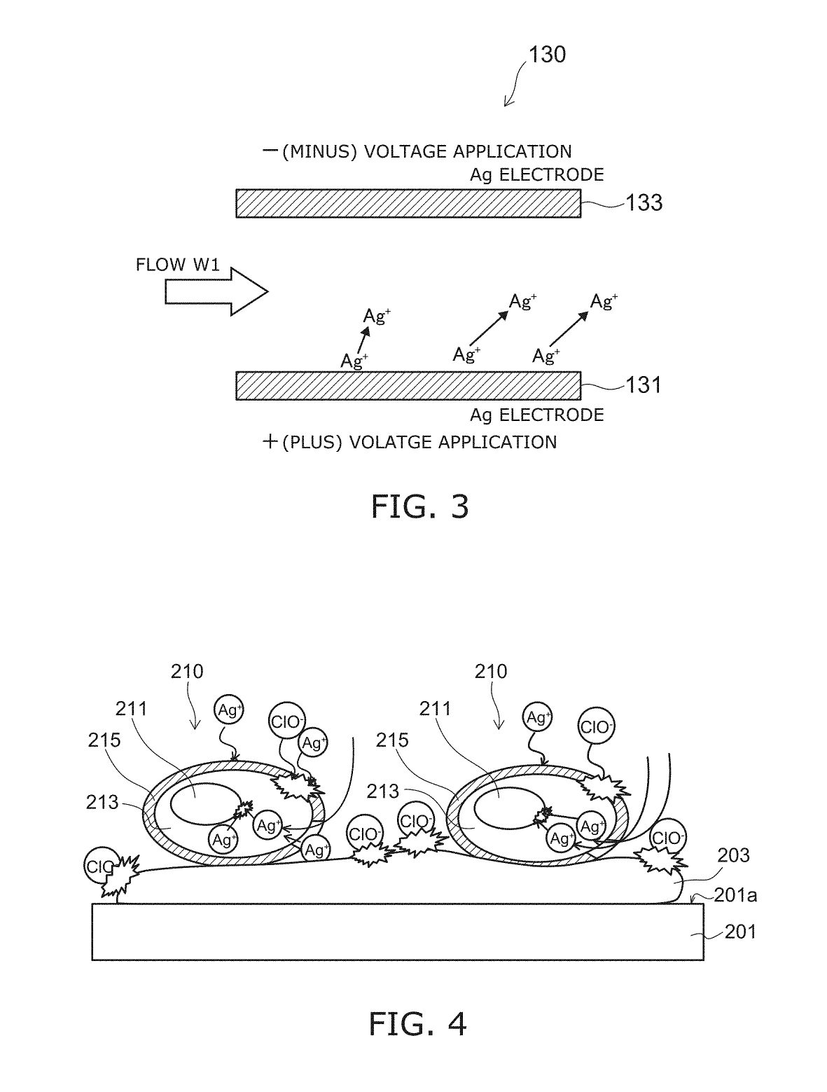

FIG. 3 is a schematic cross-sectional view showing the specific example of the sterilizing metal ionic water generating part of the embodiment.

The sterilizing metal ionic water generating part 130 of the specific example includes a pair of electrodes 131, 133, and can electrolyze the water flowing between the pair of electrodes 131, 133 by the control signal of energization outputted from the controller 145. One of the pair of electrodes 131, 132 forms the anode and the other of the pair of electrodes 131, 133 forms the cathode. The electrode forming the anode is made of silver (Ag) or a metal including silver. In general, it is favorable to form both of the pair of electrodes 131, 133 and invert polarity of the applied voltage appropriately.

If the voltage is supplied between the pair of electrodes 131, 133, silver ions are released from the electrode on the anode side (electrode 131 in FIG. 3). The released silver ions flow into the water flowing to the downstream side with flow of water W1. Thereby, The water energized in the sterilizing metal ionic water generating part 130 changes to the water containing sterilizing metal ion. The water containing sterilizing metal ion exerts, for example, sterilizing action and bacteria removing action or the like.

In the sterilizing metal ionic water generating part 130 of the embodiment, the metal ions released from the electrode 131 on the anode side are not limited to silver ions. The metal ions released from the electrode 131 on the anode side may be, for example, copper ions or zinc ions. In the following description, the case where the metal ions released from the electrode 131 on the anode side are silver ions will be described as the example.

Returning to FIG. 1, the water discharge part 143 is provided downstream side of the sterilizing metal ionic water generating part 130. The water discharge part 143 discharges at least one of the water containing hypochlorous acid or the water containing sterilizing metal ion to the object.

The motor 141 generates torque based on the signal outputted from the controller 145, and for example, rotates the water discharge part 143 about a prescribed axis. Thereby, the water discharge part 143 can discharge at least one of the water containing hypochlorous acid or the water containing sterilizing metal ion in a wider range.

The operation part 147 sends, for example, the signal based on the operation of the user to the controller 145.

The controller 145 controls the operations of the solenoid valve 103, the hypochlorous acid containing water generating part 110, sterilizing metal ionic water generating part 130, and the motor 141.

In the bacteria removing water generation device 100 according to the embodiment, when the concentration of hypochlorous acid in the water containing hypochlorous acid (hereinafter, simply referred to as “concentration of hypochlorous acid”) is relatively low, the concentration of sterilizing metal ion in the water containing sterilizing metal ion (hereinafter, simply referred to as “concentration of sterilizing metal ion”) is relatively high.

For example, in the case where a first concentration of hypochlorous acid is lower than a second concentration of hypochlorous acid, and a third concentration of sterilizing metal ion is lower than a fourth concentration of sterilizing metal ion, when the concentration of hypochlorous acid is the first concentration the concentration of sterilizing metal ion is the third concentration.

This detail will be described with reference to the drawing.

FIG. 4 is a schematic cross-sectional view showing an action of water containing hypochlorous acid and water containing sterilizing metal ion.

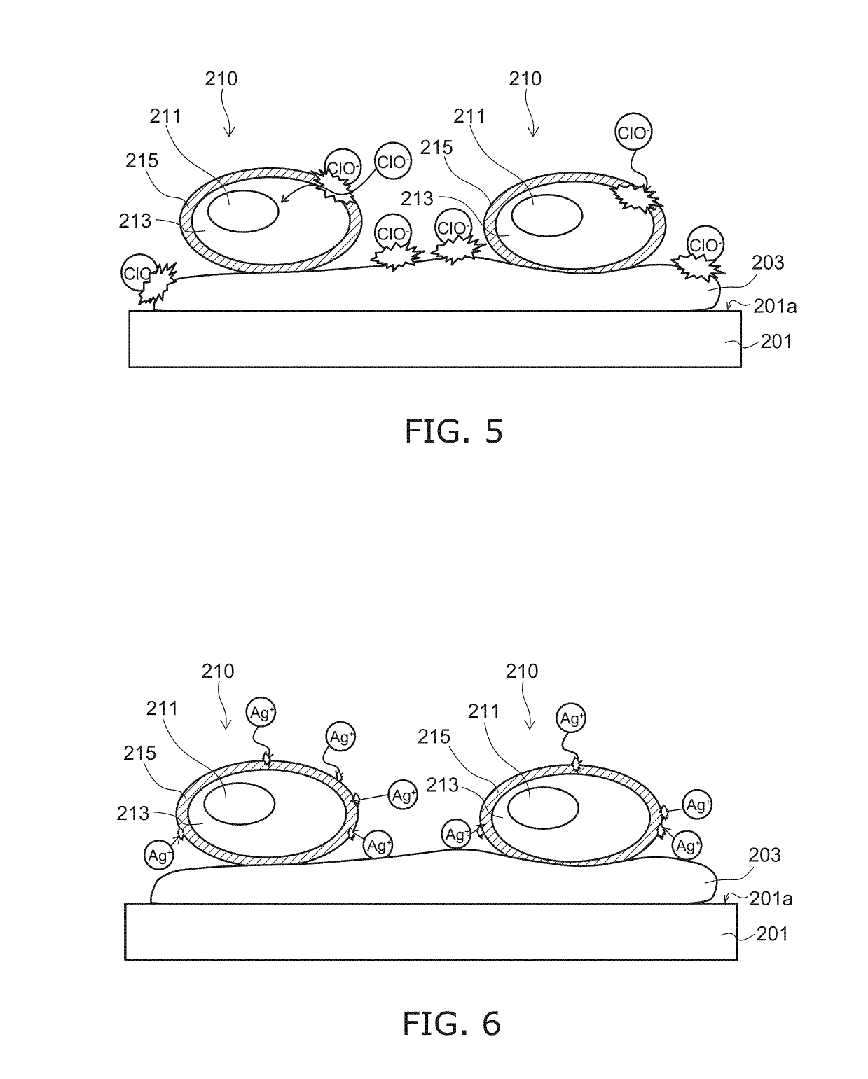

FIG. 5 is a schematic cross-sectional view showing an action of the water containing hypochlorous acid.

FIG. 6 is a schematic cross-sectional view showing an action of the water containing sterilizing metal ion.

As shown in FIG. 4 to FIG. 6, a state in which dirt 203 is attached on a surface 201a of a basic material (object) 201 will be described. The basic material 201 includes, for example, a wash place floor of a bath room. The dirt 203 includes, for example, sebum and protein or the like. The dirt 203 includes bacteria 201. The bacteria 201 have a nucleus (DNA) 211, a cytoplasm 213, a cell membrane 215.

As shown in FIG. 5, the water containing hypochlorous acid has an immediate effect, and can destroy the cell membrane 215 of the bacteria 210 in a short time, on the other hand, the water containing hypochlorous acid is easy to react dirt extremely. Therefore, a concentration of hypochlorous acid decreases because the water containing hypochlorous acid reacts with the dirt 203 before attacking the nucleus 211 of the bacteria 210.

In contrast, as shown in FIG. 6, an immediate effect of the water containing sterilizing metal ion (silver ions in FIG. 6) is lower than the immediate effect of the water containing hypochlorous acid. Therefore, the water containing sterilizing metal ion cannot destroy the cell membrane 215 of the bacteria 210 in a short time. On the other hand, the water containing sterilizing metal ion is hard to react with the dirt 203 in comparison with the water containing hypochlorous acid and enters cytoplasm 213 through the cell membrane 215 to suppress cell division of the nucleus 211. Therefore, a concentration of sterilizing metal ion lasts over a long time.

In this way, the water containing hypochlorous acid and the water containing sterilizing metal ion complement and comply with each other. Therefore, as shown in FIG. 4, the water containing hypochlorous acid destroys the cell membrane 215 of the nucleus 210 in a short time, and the water containing sterilizing metal ion (silver ions in FIG. 4) enters the inside of the bacteria 210 from the cell membrane 215 destroyed by the water containing hypochlorous acid, and then can attack the nucleus 211 of the bacteria 210 under a condition of lasting the concentration over a long time.

Here, the concentration of hypochlorous acid is proportional to the amount of chlorine ion (Cl⁻) contained in the tap water. Therefore, in the case where the amount of chlorine ion is small, the concentration of hypochlorous acid decreases, and destruction of the cell membrane of the bacteria may be insufficient. In the case where the destruction of the cell membrane of the bacteria is insufficient, there is a problem that the water containing sterilizing metal ion cannot arrive at the nucleus of the bacteria and the bacteria removing effect is insufficient.

In contrast, in the bacteria removing water generation device 100 according to the embodiment, when the concentration of hypochlorous acid is relatively low, the concentration of sterilizing metal ion is relatively high.

For example, in the case where the concentration of hypochlorous acid is detected to be low, the controller 145 controls the voltage supplied to the sterilizing metal ionic water generating part 130 and increases the concentration of sterilizing metal ion.

For example, the concentration of chlorine ion contained in the tap water is detected by detecting the voltage applied to the hypochlorous acid containing water generating part 110. Specifically, a voltage between the anode 111 of the hypochlorous acid containing water generating part 110 and the cathode of the hypochlorous acid containing water generating part 110 is detected. Based on the voltage, water quality of the tap water (for example, electric conductivity of the water flowing into the hypochlorous acid containing water generating part 110) is detected. Thereby, the concentration of chlorine ion contained in the tap water can be detected. The controller 145 controls the voltage supplied to the sterilizing metal ionic water generating part 130 based on the detected concentration of chlorine ion. The concentration of chlorine ion contained in the tap water can be detected by the similar method described above by using the sterilizing metal ionic water generating part 130.

The bacteria removing water generation device 100 according to the embodiment is an ion concentration detecting part provided between the hypochlorous acid containing water generating part 110 and the negative pressure breaking device 121, and may include the ion concentration detecting part detecting the concentration of chlorine ion contained in the tap water.

As previously described in FIG. 4 to FIG. 6, hypochlorous acid is generated from chlorine ion contained in the tap water as a raw material. Therefore, when the amount of chlorine ion contained in the tap water is relatively low, the concentration of hypochlorous acid is relatively low. On the other hand, sterilizing metal ion is generated from the electrode eluted. Therefore, the concentration of sterilizing metal ion does not depend on the amount of chlorine ion contained in the tap water. However, the generation amount of sterilizing metal ion is limited.

According to the bacteria removing water generation device 100 according to the embodiment, when the concentration of hypochlorous acid is relatively low, the concentration of sterilizing metal ion is relatively high. Thereby, even if the concentration of chlorine ion contained in the tap water is relatively low and the concentration of hypochlorous acid is relatively low, a sufficient bacteria removing effect can be exerted. On the other hand, when the concentration of hypochlorous acid is relatively high, the concentration of sterilizing metal ion is relatively low. Thereby, while ensuring the sufficient bacteria removing effect, a life of the electrodes of the sterilizing metal ionic water generating part 130 can be improved.

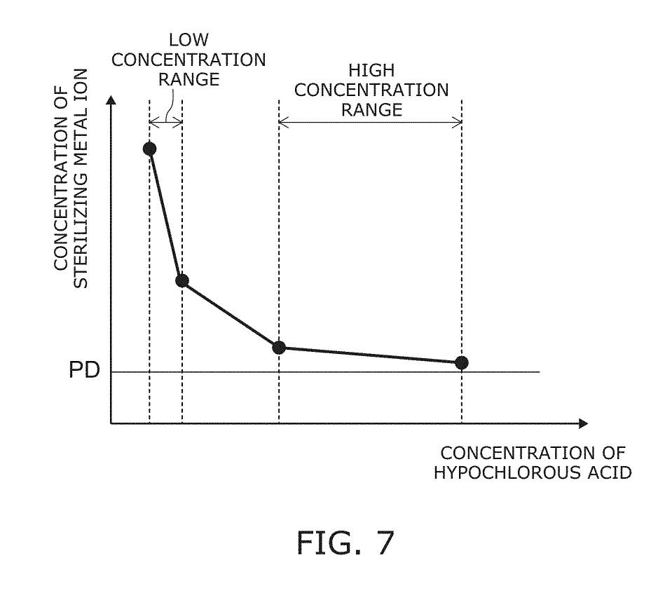

FIG. 7 is a graph view illustrating one example of the relationship between a concentration of hypochlorous acid and a concentration of sterilizing metal ion.

A horizontal axis of the graph view shown in FIG. 7 represents the concentration of hypochlorous acid. A vertical axis of the graph view shown in FIG. 7 represents the concentration of sterilizing metal ion.

When the concentration of hypochlorous acid is relatively high, the water containing hypochlorous acid can destroy the cell membrane 215 sufficiently. On the other hand, the effect of the water containing hypochlorous acid to attack the nucleus 211 of the bacteria 210 is lower than the effect of the water containing sterilizing metal ion to attack the nucleus 211 of the bacteria 210.

In contrast, as shown in FIG. 7, in the bacteria removing water generation device 100 according to the embodiment, when the concentration of hypochlorous acid is relatively high (high concentration range), a change rate of the concentration of sterilizing metal ion is relatively low. Thereby, the effect of the water containing sterilizing metal ion to attack the nucleus 211 of the bacteria 210 can be obtained.

When the concentration of hypochlorous acid is comparatively low, the water containing hypochlorous acid may not sufficiently destroy the cell membrane 215. In such a case, the water containing sterilizing metal ion is also required to destroy the cell membrane 215 of the bacteria 210.

In contrast, as shown in FIG. 7, in the bacteria removing water generation device 100 according to the embodiment, when the concentration of hypochlorous acid is relatively low (low concentration range), a change rate of the concentration of sterilizing metal ion is relatively high. Thereby, even if the concentration of hypochlorous acid is relatively low, the cell membrane 215 can be destroyed sufficiently.

As previously described with regard to FIG. 4 to FIG. 6, the water containing hypochlorous acid is likely to react with the dirt 203 in comparison with the water containing sterilizing metal ion. Therefore, even if the concentration of hypochlorous acid is high, the effect of the water containing hypochlorous acid to attack the nucleus 211 of the bacteria 210 is lower than the effect of the water containing sterilizing metal ion to attack the nucleus 211 of the bacteria 210. When the concentration of sterilizing metal ion is lower than a prescribed concentration, the effect of the water containing sterilizing metal ion to attack the nucleus 211 of the bacteria 210 is low.

In contrast, as shown in FIG. 7, the concentration of sterilizing metal ion is higher than a prescribed concentration PD even if the concentration of hypochlorous acid is relatively high. Thereby, even if the concentration of hypochlorous acid is high, the concentration of sterilizing metal ion is maintained to be a concentration higher than the prescribed concentration PD. The prescribed concentration PD is the concentration of sterilizing metal ion maintaining the effect of the water containing sterilizing metal ion to attack the nucleus 211 of the bacteria 210. Therefore, the effect of the water containing sterilizing metal ion to attack the nucleus 211 of the bacteria 210 is maintained.

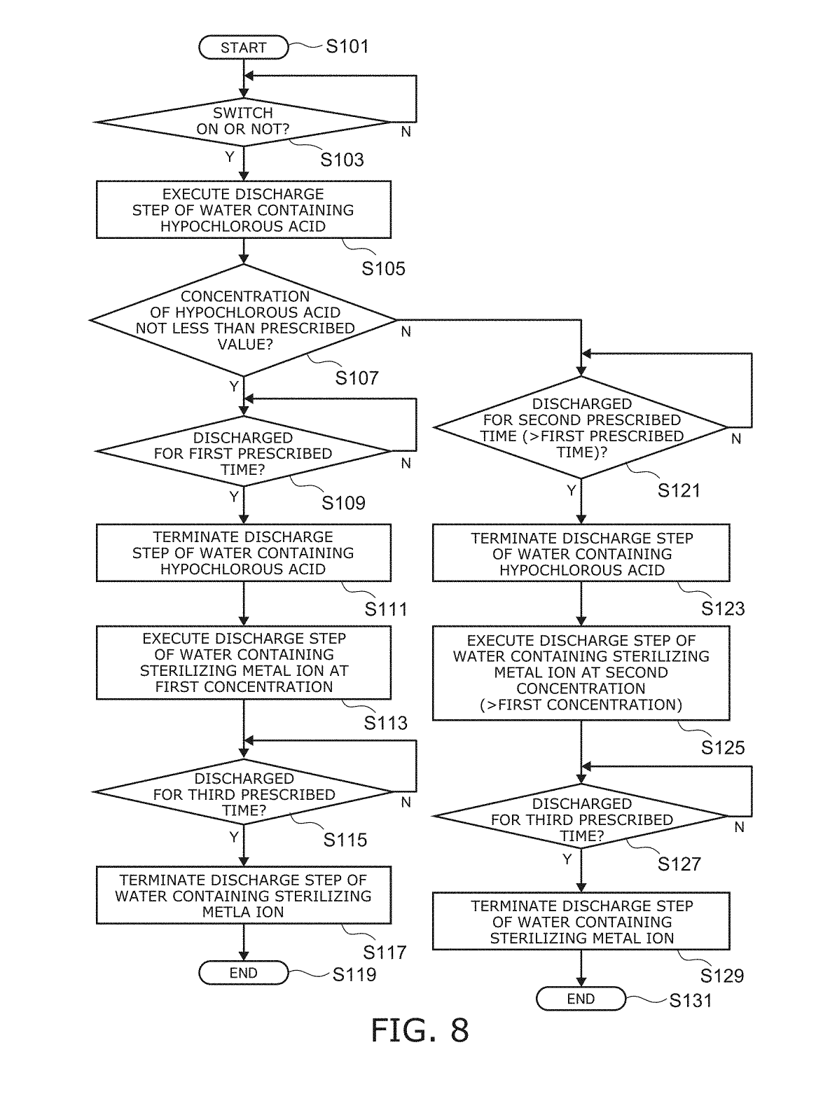

FIG. 8 is a flow chart showing an operation of the bacteria removing water generation device according to the embodiment.

In a state before the start of the operation of the bacteria removing water generation device 100, the solenoid valve 103 is closed, the motor 141 stops, and the voltage is not supplied to the hypochlorous acid containing water generating part 110 and the sterilizing metal ionic water generating part 130.

If the bacteria removing water generation device 100 starts the operation (step S101), it is determined whether a switch is turned on or not (step S102). In the case where the switch is not turned on (step S103:N), the bacteria removing water generation device 100 determines subsequently whether the switch is turned on or not (step S103). In the case where the switch is turned on, (step S103:Y), the water discharge part 143 discharges the water containing hypochlorous acid (step S105). At this time, the solenoid valve 103 opens, the motor 141 starts the operation, and the voltage is supplied to the hypochlorous acid containing water generating part 110.

Subsequently, the bacteria removing water generation device 100 determines whether the concentration of hypochlorous acid is not less than the prescribed concentration or not (step S107). In the case where the concentration of hypochlorous acid is not less than the prescribed concentration (step S107:Y), the bacteria removing water generation device 100 determines whether the water discharge part 143 discharges the water containing hypochlorous acid for a first prescribed time or not (step S109). In the case where the water discharge part 143 does not discharge the water containing hypochlorous acid for the first prescribed time (step S109:N), the bacteria removing water generation device 100 determines subsequently whether the water discharge part 143 discharges the water containing hypochlorous acid for the first prescribed time or not (step S109). In the case where the water discharge part 143 discharges the water containing hypochlorous acid for the first prescribed time (step S109:Y), the water discharge part 143 terminates the discharge of the water containing hypochlorous acid (step S111). At this time, the supply of the voltage to the hypochlorous acid containing water generating part stops.

Subsequently, the water discharge part 143 discharges water containing sterilizing metal ion of a first concentration (step S113). At this time, a voltage is supplied to the sterilizing metal ionic water generating part 130. Subsequently, the bacteria removing water generation device 100 determines whether the water discharge part 143 discharges the water containing sterilizing metal ion of the first concentration for a third prescribed time (step S115). In the case where the water discharge part 143 does not discharge the water containing sterilizing metal ion of the first concentration for the third prescribed time (step S115:N), the bacteria removing water generation device 100 determines subsequently whether the water discharge part 143 discharges the water containing sterilizing metal ion of the first concentration for the third prescribed time or not (step S115). In the case where the water discharge part 143 discharges the water containing sterilizing metal ion of the first concentration for the third prescribed time (step S115:Y), the water discharge part 143 terminates the discharge of the water containing sterilizing metal ion of the first concentration (step S117). Subsequently, the bacteria removing water generation device 100 terminates the operation (step S119).

On the other hand, in the case where the concentration of hypochlorous acid is not less than the prescribed concentration (step S107:N), the bacteria removing water generation device 100 determines whether the water discharge part 143 discharges the water containing hypochlorous acid for a second prescribed time or not (step (S121). Here, the second prescribed time is longer than the first prescribed time (see step S109). In the case where the water discharge part 143 does not discharge the water containing hypochlorous acid for the second prescribed time (step S121:N), the bacteria removing water generation device 100 determines whether the water discharge part 143 discharges the water containing hypochlorous acid for the second prescribed time or not (step S121). In the case where the water discharge part 143 discharges the water containing hypochlorous acid for the second prescribed time (step S121:Y), the water discharge part 143 terminates the discharge of the water containing hypochlorous acid (step S123). At this time, the supply of the voltage to the hypochlorous acid containing water generating part stops.

Subsequently, the water discharge part 143 discharges water containing sterilizing metal ion of a second concentration (step S125). Here, the second concentration is higher than the first concentration (see step S113). At this time, a voltage is supplied to the sterilizing metal ionic water generating part 130. Subsequently, the bacteria removing water generation device 100 determines whether the water discharge part 143 discharges the water containing sterilizing metal ion of the second concentration for the third prescribed time or not (step S127). In the case where the water discharge part 143 does not discharge the water containing sterilizing metal ion of the second concentration for the third prescribed time (step S127:N), the bacteria removing water generation device 100 determines subsequently whether the water discharge part 143 discharges the water containing sterilizing metal ion of the second concentration for the third prescribed time or not (step S127). In the case where the water discharge part 143 discharges the water containing sterilizing metal ion of the second concentration for the third prescribed time (step S127:Y), the water discharge part 143 terminates the discharge of the water containing sterilizing metal ion of the second concentration (step S129). Subsequently, the bacteria removing water generation device 100 terminates the operation (step S131).

In this way, in the bacteria removing water generation device 100 according to the embodiment, in the case where the concentration of hypochlorous acid is relatively low (step S107:N), the water discharge part 143 discharges the water containing hypochlorous acid for a relatively long time (step S121). In the bacteria removing water generation device 100 according to the embodiment, an instantaneous flow rate (flow rate per unit time) of the water containing hypochlorous acid in the case where the concentration of hypochlorous acid is relatively low is the same as an instantaneous flow rate of the water containing hypochlorous acid in the case where the concentration of hypochlorous acid is relatively high.

Thereby, in the case where the concentration of hypochlorous acid is relatively low, the water discharge part 143 discharges the water containing a relatively large amount of hypochlorous acid. According to this, even if the concentration of hypochlorous acid is relatively low, the cell membrane 215 of the bacteria 210 can be more certainly destroyed. Since the instantaneous flow rate of the water containing hypochlorous acid is constant, even if the concentration of hypochlorous acid is relatively low, while maintaining a state of higher concentration of hypochlorous acid, the water containing a larger amount of hypochlorous acid can be discharged. Thereby, the cell membrane 215 of the bacteria 210 can be more certainly destroyed.

For example, in the case where the concentration of hypochlorous acid is relatively low, discharging of the water containing a relatively large amount of hypochlorous acid by the water discharge part 143 may be executed by the user operating the operation part 147. For example, the user can select a mode in which the water discharge part 143 discharges the water containing the relatively large amount of hypochlorous acid in the case where the concentration of hypochlorous acid is relatively low.

In the bacteria removing water generation device 100 according to the embodiment, a discharge time of the water containing sterilizing metal ion in the case where the concentration of hypochlorous acid is relatively low is the same as a discharge time of the water containing sterilizing metal ion in the case where the concentration of hypochlorous acid is relatively high (step S115. step S127). In the bacteria removing water generation device 100 according to the embodiment, the instantaneous flow rate of the water containing sterilizing metal ion in the case where the concentration of hypochlorous acid is relatively low is the same as the instantaneous flow rate of the water containing sterilizing metal ion in the case where the concentration of hypochlorous acid is relatively high.

Thereby, a discharge amount of the water containing sterilizing metal ion in the case where the concentration of hypochlorous acid is relatively low is the same as a discharge amount of the water containing sterilizing metal ion in the case where the concentration of hypochlorous acid is relatively high. Therefore, an increasing rate of the discharge amount of the water containing sterilizing metal ion in the case where the concentration of hypochlorous acid is relatively low is lower than an increasing rate of the discharge amount of the water containing hypochlorous acid in the case where the concentration of hypochlorous acid is relatively low (step S109, step S121, step S115, step S127).

As previously described in FIG. 4 to FIG. 6, the immediate effect of the water containing sterilizing metal ion is lower than the immediate effect of the water containing hypochlorous acid. Therefore, if the discharge amount of the water containing hypochlorous acid is increased, a certain effect is obtained, however even if the discharge amount of the water containing sterilizing metal ion is increased, it is impossible to obtain an effect as much as the effect obtained when the discharge amount of the water containing hypochlorous add is increased.

According to this, while ensuring the sufficient bacteria removing effect, the life of the electrodes of the sterilizing metal ionic water generating part 130 can be improved.

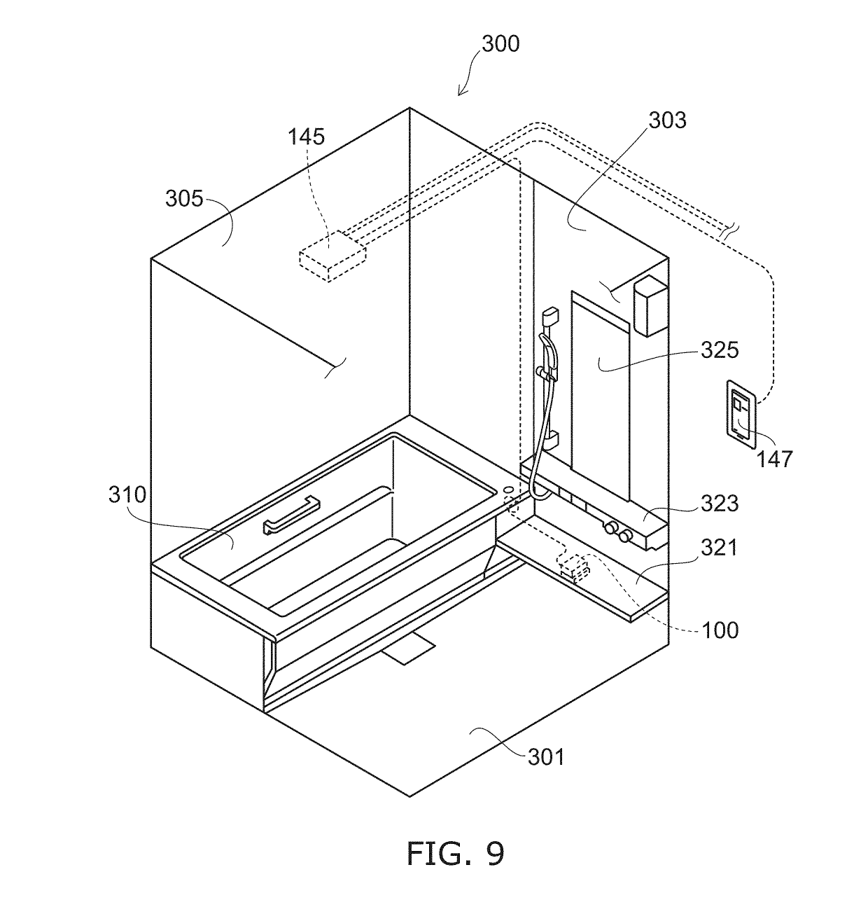

FIG. 9 is a schematic cross-sectional view showing a bath room including the bacteria removing water generation device according to the embodiment.

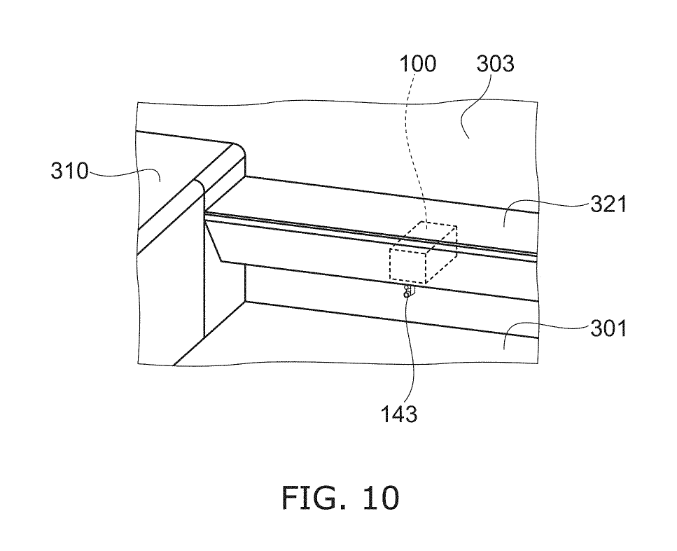

FIG. 10 is a schematic enlarged view shown by enlarging a portion where the bacteria removing water generation device is provided.

A bath room 300 shown in FIG. 9 includes a wash place floor 301, a bathtub 301, a first wall panel 303, a second wall panel 305, and the bacteria removing water generation device 100. In the bath room 300 shown in FIG. 9, the controller 145 is provided on a ceiling back of the bath room 300. However, the location of the controller 145 is not limited to the ceiling back of the bath room 300, but may be provided integrally with the bacteria removing water generation device 100.

The first wall panel is provided with a bath room counter 321, a faucet counter 323, and a mirror 325. As shown in FIG. 10, a portion of the bacteria removing water generation device 100 is provided inside the bath room counter 321. The water discharge part 143 extends to the outside of the bath room counter 321 through a lower surface of the bath room counter 321. The water discharge part 143 discharges at least one of the water containing hypochlorous add and the water containing sterilizing metal ion to the wash place floor 301 (object).

The operation part 147 is provided outside the bath room 300. The operation part may be provided inside the bath room 300. Or, the operation part 147 may be provided integrally with the bacteria removing water generation device 100. For example, the user can select a mode in which the water discharge part 143 discharges the water containing the relatively large amount of hypochlorous acid in the case where the concentration of hypochlorous acid is relatively low.

The bacteria removing water generation device 100 is as described previously in FIG. 1 to FIG. 8. According to this, even if in the bath room 300 including the bacteria removing water generation device 100, the concentration of chlorine ion contained in the tap water is relatively low and the concentration of hypochlorous acid is relatively low, the sufficient bacteria removing effect can be exerted.

Hereinabove, the embodiments of the invention are described. However, the invention is not limited to the descriptions. Design modification appropriately made by a person skilled in the art in regard to the embodiments described above is within the scope of the invention to the extent that the features of the invention are included. For example, the shape, the dimension, the material, the disposition or the like of the components included in the bacteria removing water generation device 100 or the like and the placement configuration or the like of the bacteria removing water generation device 100 are not limited to illustrations and can be changed appropriately.

The location of the bacteria removing water generation device 100 is not limited to the bath room 300. For example, the bacteria removing water generation device 100 may be located in a system kitchen or a washstand or the like.

Further, any two or more components of the embodiments may be combined within the extent of technical feasibility and are included in the scope of the invention to the extent that the purport of the invention is included.

### INDUSTRIAL APPLICABILITY

According to an aspect of the invention, a bacteria removing water generation device which is capable of exerting a sufficient bacteria removing effect even if a concentration of chlorine ion contained in tap water is low is provided.

### EXPLANATION OF LETTERS OR NUMERALS

100 bacteria removing water generation device
101 stopcock
103 solenoid valve
105 pressure regulator valve
107 check valve
110 hypochlorous acid containing water generating part
111 anode
113 cathode
121 negative pressure breaking device
130 sterilizing metal ionic water generating part
131, 133 electrode
141 motor
143 water discharge part
145 controller
147 operation part
151 flow channel
201 basic material
201a surface
203 dirt
210 bacteria
211 nucleus
213 cytoplasma
215 cell membrane
300 bath room
301 wash place floor
303 first wall panel
305 second wall panel
310 bathtub
321 bath room counter
323 faucet counter
325 mirror

1. A bacteria removing water generation device comprising:
a hypochlorous acid containing water generating part generating water containing hypochlorous acid by electrolyzing tap water;
a sterilizing metal ionic water generating part including electrodes, sterilizing metal ion being eluted from the electrodes into the tap water by electrolyzing tap water;
a water discharge part discharging at least one of the water containing the hypochlorous acid and water containing the sterilizing metal ion; and
a controller controlling operations of the hypochlorous acid containing water generating part and the sterilizing metal ionic water generating part,
when a concentration of the hypochlorous acid in the water containing the hypochlorous acid is relatively low, a concentration of the sterilizing metal ion in the water containing the sterilizing metal ion being relatively high.

2. The bacteria removing water generation device according to claim 1, wherein
when the concentration of the hypochlorous acid is relatively low, a change rate of the concentration of the sterilizing metal ion is relatively high.

3. The bacteria removing water generation device according to claim 1, wherein
the concentration of the sterilizing metal ion is higher than a prescribed concentration even if the concentration of the hypochlorous acid is high.

4. The bacteria removing water generation device according to claim 1, wherein
the water discharge part discharges the water containing a relatively large amount of hypochlorous acid when he concentration of hypochlorous acid is relatively low.

5. The bacteria removing water generation device according to claim 4, wherein
an instantaneous flow rate of the water containing the hypochlorous acid when the concentration of the hypochlorous acid is relatively low is the same as an instantaneous flow rate of the water containing the hypochlorous acid when the concentration of the hypochlorous acid is relatively high.

6. The bacteria removing water generation device according to claim 5, wherein
an increasing rate of a discharge amount of the water containing the sterilizing metal ion when the concentration of the hypochlorous acid is relatively low is lower than an increasing rate of a discharge amount of the water containing the hypochlorous acid when the concentration of the hypochlorous acid is relatively low.

7. The bacteria removing water generation device according to claim 4, further comprising:
an operation part, when the concentration of the hypochlorous acid is relatively low, in the operation part a mode being selectable, in the mode the water discharge part discharging the water containing the relatively large amount of the hypochlorous acid.
